(19) United States
(12) Patent Application Publication
(10) Pub. No.: US 2009/0306593 A1
(43) Pub. Date: Dec. 10, 2009

(54) Mechanically operated liquid pump

(76) Inventors: Roland Wex, Melsungen (DE); Harald Hansmann, Wismar (DE); Christian Kenzler, Pritzwalk (DE)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/989,253

(22) PCT Filed: Jul. 21, 2006
(86) PCT No.: PCT/EP2006/007191

§ 371 Date: Mar. 18, 2009

(30) Foreign Application Priority Data
Jul. 22, 2005 (EP) 05 015 967.2

(51) Int. Cl. A61M 5/152

(52) U.S. Cl. 604/132

(57) ABSTRACT

The invention relates to a mechanically operated liquid pump (1), particularly for medical or nutrition-physiological liquids, with a housing (2) and with an expandable clastic element (16), which is held therein and which serves to store and deliver the liquid, and with a closeable access (21) and exit (50) for the liquid from the elastic element as well as with a device for largely constantly maintaining or limiting the liquid volume flow discharged by the elastic element. According to the invention, the following features are provided: the elastic element is provided in the form of a balloon (16) that has an opening (17a); a core (11) is housed inside the housing and is inserted into the balloon via the opening; means (20) for fastening and sealing the balloon in the area of its end having the opening are provided on the core or on the housing, and; when relatively unstressed, the balloon rests against the core at a distance from its fastening.

### FIELD OF THE INVENTION

The invention relates to a mechanically operated liquid pump, particularly for medical or nutrient liquids, and for liquids in the biological and laboratory sector, with a housing, and with an expandable elastic element that is held therein and that serves to store and dispense the liquid, and also with a closeable inlet for the liquid traveling to the elastic element, an outlet for the liquid traveling from the elastic element, and a device for maintaining substantially constant and/or restricting the volumetric flow of liquid dispensed from the elastic element.

### BACKGROUND OF THE INVENTION

Many different types of liquid pumps are used for conveying medical, nutrient or biological liquids, or liquids in the laboratory sector. For example, pumps are known that are operated by electric energy, electrochemically, by gas, mechanically, electromechanically, and by physical mechanics. Many of these pumps can generally only be used after quite a long start-up time and often provide the user and consumer with insufficient dosing precision. The known pump systems are also very expensive and are not very satisfactory from the environmental point of view. They often cannot be used portably by the user.

Mechanically operated liquid pumps are characterized by a drive mechanism, which can generally be of quite simple configuration. Thus, various designs have been disclosed in which expandable elastic elements are used to store and dispense the liquid.

A mechanically operated liquid pump, particularly for medical or nutrient liquids, is known from EP 0 944 405 B1. In said pump, the expandable elastic element is designed as a hose. The latter is arranged inside a tube, which is closed in the area of one end by means of a stationary end cap. The hose is mounted with its one open end in a sealed manner in this end cap. A conduit, which extends through the end cap, leads into the hose end. Remote from this end cap, another end cap is mounted displaceably in the tube and receives the hose in the area of its other open end. There, the hose is correspondingly mounted, thus sealed off in this displaceable end cap, and a conduit is passed through this end cap and is guided into the hose. The conduit assigned to the movable end cap is allocated a valve through which the liquid can flow into the hose, whereupon the hose is expanded until it bears on the inside wall of the tube. On account of the expansion of the hose, the movable end cap is also moved away from the stationary end cap. By means of the elastic restoring force of the hose, acting radially inward, the liquid is guided through the conduit assigned to the stationary end cap and delivered to the patient.

Such a configuration of the mechanically operated liquid pump is complex, because an elastic element is used inside a hose, which therefore has two diametral openings to which separate conduits are assigned, one having the function of the inlet, the other having the function of the outlet. Besides this, the liquid cannot be dispensed completely from the hose and thus delivered to the patient, the reason being that, as the hose walls come increasingly closer as a result of the liquid being dispensed to the patient, the elastic restoring forces of the hose decline. Finally, the hose expands radially outward in a uniform manner until it bears on the inside wall of the tube. Because of the defined rotationally symmetrical design of the tube, the pump has an unfavorable dimension in width and depth. A flat pump is therefore not possible, on account of the design of the elastic element in the form of a hose. The sealing of the hose is complicated, since it has to be done at two places.

A mechanically operated liquid pump of similar design is described in EP 0 424 494994 B1.

Mechanically operated liquid pumps, particularly for medical or nutrient liquids, have been disclosed in which a bag is used to receive the liquid, and a conduit connected to the bag serves to dispense from the bag the liquid located in the bag. The bag interacts with a device for exerting a compressive force on the bag, for the purpose of dispensing the liquid from the latter. The device comprises an elastic element for acting on one side of the bag, and a further element for acting on the opposite side of the bag. Such a liquid pump is described in DE 199 28 131 A1. Similar mechanically operated liquid pumps based on this operating principle are described in DE 100 63 975 A1 and DE 100 58 121 A1.

A pump used particularly in the field of chromatography is described in German application 27 01 100. In said pump, a liquid, for example an elution agent, is expelled by delivery of a pressurized gas. The pump comprises a flexible container in the form of a hose-like membrane, which is sealed at one end by a screw clamp. The hose is surrounded by a tube in a sealed manner, with a space being formed between tube and hose. In use in chromatography, nitrogen introduced into this space presses the hose together, whereupon all or some of the elution agent is expelled and forced through a chromatography column.

### SUMMARY OF THE INVENTION

The object of the invention is to develop a mechanically operated liquid pump of the type mentioned at the outset, in such a way that complete dispensing of the liquid is guaranteed, while retaining a simple design of the drive mechanism of the pump.

In a liquid pump of the type mentioned at the outset, the object is achieved by the following features:

the elastic element is designed as a balloon, which is provided with an opening,

a core is mounted in the housing and is inserted into the balloon via the opening of said balloon,

means are provided by which the balloon, in the area of its end with the opening, is fastened and sealed on the core or on the housing,

in the relatively unstressed state, the balloon bears on the core at a distance from its fastening.

The drive mechanism of the mechanically operated liquid pump according to the invention is already characterized particularly by the fact that the elastic element is designed as a balloon. It is therefore an elastic element provided with a single opening. Accordingly, this opening serves both as an inlet for the liquid and also as an outlet for the liquid. A further particularity is that a core interacting with the balloon is inserted into the latter through the opening of the balloon. The balloon bears in a relatively unstressed state on the core. This is to be understood as meaning that the balloon can bear with a certain pretensioning on the core, since complete emptying of the balloon is to be sought. The balloon is fastened and sealed exclusively in the area of its end that has the opening. This is preferably done directly on the core. It is also conceivable for the fastening and sealing of the balloon to be done on the housing.

The balloon can in principle be made of any material having the necessary elasticity for storing and dispensing the liquid. Silicone is regarded as the preferred material. The balloon is intended to have a capacity of 10 ml to 150 ml in particular.

In the liquid pump, it is considered particularly important for it to have a relatively flat design, since it is generally worn by a patient directly on the body. A liquid pump with a circular diameter is not particularly desirable in this respect.

To achieve an expansion of the balloon preferably in its direction of width and less so in the direction of its height, a development of the invention is proposed in which, in a first direction of extent perpendicular to the longitudinal axis of the core, the balloon has relatively thick wall portions, and, in a second direction of extent perpendicular to the longitudinal axis of the core and perpendicular to the first direction of extent, it has relatively thin wall portions. This means that the balloon deforms more strongly in the area of the thin wall portions, with the result that, upon expansion of the element, a cross-sectional configuration approximating substantially to the shape of an ellipse is obtained. The balloon is produced in particular by injection molding.

According to a preferred development of the invention, a cap can be connected to the housing, in particular a cap that is kidney-shaped in cross section, seen transverse to the longitudinal axis of the core. The balloon is arranged inside this cap. The kidney-shaped configuration of the cap means that it able to adapt snugly to the physical contour of the person wearing the pump in the waist region. During its expansion, the balloon configured with wall portions of different thickness approximately follows the inner contour of the thin-walled kidney-shaped cap. When the balloon is filled sufficiently with liquid, it is able to bear on the inside wall of the cap. The width/height ratio of the cap is relatively great, preferably 1.5:1 to 3:1, in particular approximately 2:1. The cap can preferably be clipped nonreleasably onto the housing.

In a preferred embodiment, the core is rotationally symmetrical, in particular cylindrical. It thus has a constant diameter, apart from a possible thickening in the area of connection to the housing. Correspondingly, the balloon likewise has a cylinder shape in the unstressed state, that is to say when independent of the core, the diameter of the cylinder being smaller than the diameter of the core, relative to the area of the core with which the balloon comes into contact in the relatively unstressed state. It is considered particularly advantageous if the balloon can bear completely on the core, such that no liquid is then located between balloon and core.

The core can be designed in different ways for introducing the liquid into the balloon and for dispensing the liquid from the balloon. Preferably, a channel forming part of the inlet and outlet extends through the core. The channel extends through the core particularly in its longitudinal axis and in its radial direction. Starting from the area of the channel extending on the longitudinal center axis, liquid can pass into the radial portions of this channel, such that these axially and radially extending channel portions provide a connection to the interior of the balloon. The length/diameter ratio of the core is 3:1 to 8:1, in particular 5:1 to 6:1.

The means for fastening and sealing the balloon are preferably designed as a sleeve which, with the balloon pulled onto the core, clamps the latter between the sleeve and the core.

Further features of the invention are set forth in the dependent claims, in the description of the figures, and in the figures themselves. It will be noted that all the individual features and all combinations of the individual features are part of the invention.

The invention is depicted in the figures in one illustrative embodiment, without being limited to this.

### BRIEF DESCRIPTION OF THE DRAWINGS

FIG. 1 shows an exploded view of the mechanically operated liquid pump according to the invention,

FIG. 2 shows a vertical longitudinal midsection through the pump shown in FIG. 1, with the balloon bearing on the core,

FIG. 3 shows a section according to FIG. 2, with the balloon filled with liquid,

FIG. 4 shows a vertical longitudinal section through the pump shown in FIG. 1, at a distance from the longitudinal center axis of the pump, in the area of a valve of the pump,

FIG. 5 shows a section, cut transversely through the pump illustrated in FIG. 1, in the area of the valve,

FIG. 6 shows a horizontal longitudinal midsection through the pump shown in FIG. 1, with the balloon bearing on the core,

FIG. 7 shows a section according to FIG. 6, with the balloon filled with liquid,

FIG. 8 shows a section through the pump, cut transversely in the area where the core is supported,

FIG. 9 shows a section through the pump, cut transversely in the area of the unsupported portion of the core and of the liquid-filled balloon,

FIG. 10 shows an enlarged sectional view of core, balloon and clamping ring for connection of balloon and core, with the balloon bearing on the core,

FIG. 11 shows a section, cut transversely to the longitudinal extent of the core, through the core and the balloon, with the balloon bearing on the core,

FIG. 12 shows a sectional view according to FIG. 11 for a modified cross-sectional configuration of the balloon,

FIG. 13 shows an enlarged sectional view of the valve shown in FIG. 4, and

FIG. 14 shows a diagram illustrating the operating principle of the mechanically operated liquid pump, and indicating physical parameters.

### DETAILED DESCRIPTION OF THE PREFERRED EMBODIMENTS

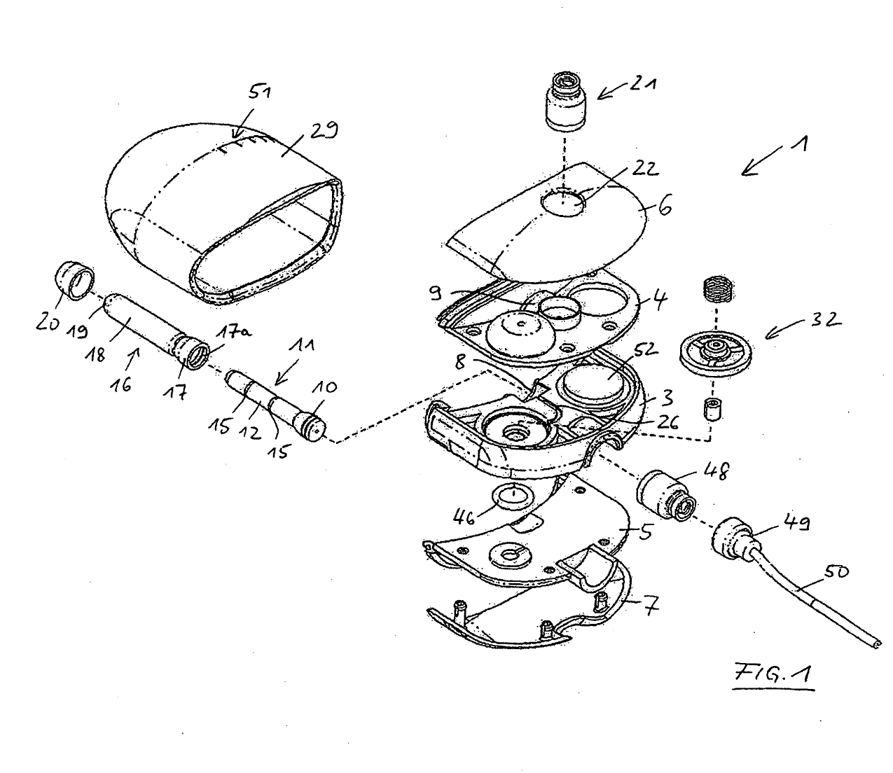

For purposes of description herein, the terms “upper”, “lower”, “right”, “left”, “rear”, “front”, “vertical”, “horizontal” and derivatives thereof shall relate to the invention as oriented in FIG. 1. However, it is to be understood that the invention may assume various alternative orientations and step sequences, except where expressly specified to the contrary. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

The mechanically operated liquid pump 1 illustrated in FIG. 1 is used in particular for administering medical or nutrient liquids, for example for administering a liquid medicament.

The pump 1 has a multi-component housing 2 formed by a middle part 3, by an upper part 4 and a lower part 5 that interact with said middle part 3, by an upper shell 6 interacting with the upper part, and by a lower shell 7 interacting with the lower part 5.

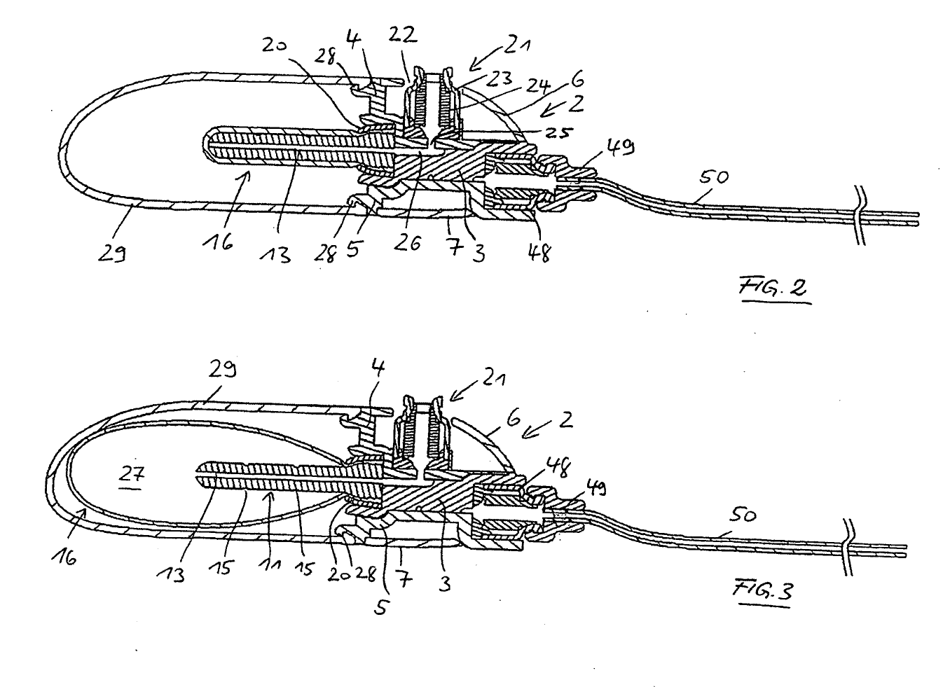

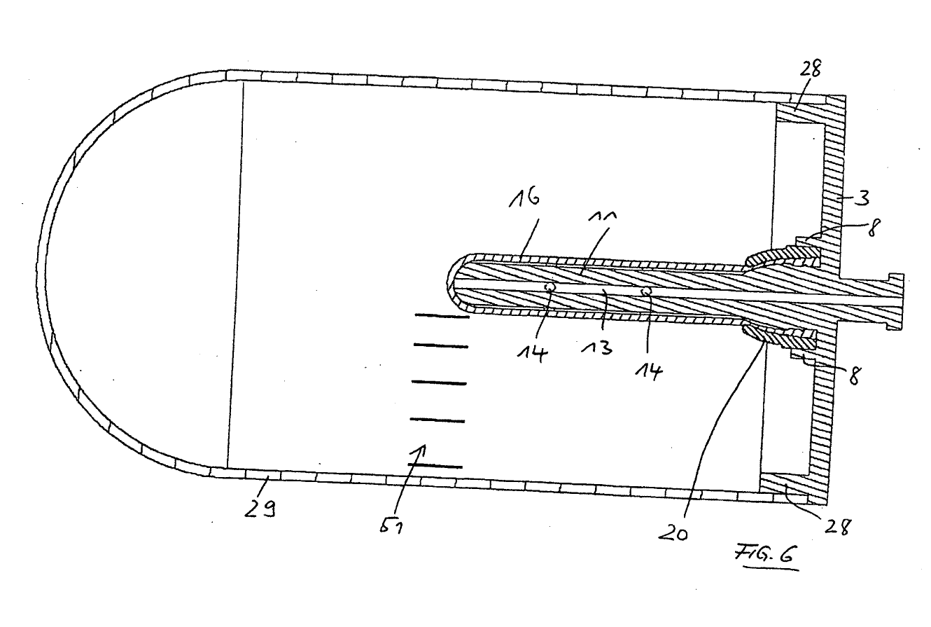

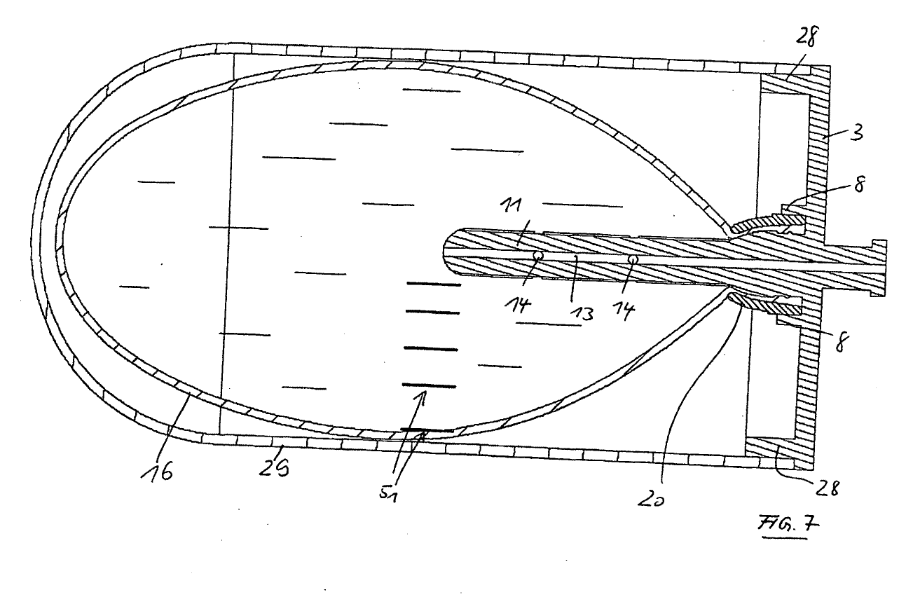

The middle part 3 is provided on its upper face with a recess 8 that is open to the free edge of the middle part 3 and that has a semicircular cross section, and the upper part 4 is provided on its lower face, and in the corresponding edge area, with a corresponding semicircular recess 9. With the upper part 4 connected to the middle part 3, the two recesses 8 and 9 form a circular cross section for receiving a conically widened end area 10 of a core 11. Except at its end area 10, the core 11 has a constant external diameter. This cylindrical portion of the core 11 is designated by reference number 12. A channel 13 (see FIG. 2) extends through the core 11 along its longitudinal center axis, and several channels 14 extending radially through the core 11 branch off from the channel 13 in the area of the portion 12 (FIG. 6). In the area of the outer circumference of the core 10, the radial channels 14 open into circumferential grooves 15 of the core 11.

An elastic element interacts with the core 11 and is designed as a silicone balloon 16. The latter is produced by injection molding. The balloon has a conically widened end area 17 with opening 17a, corresponding to the end area 10 of the core 11, and it has a portion 18 which corresponds to the outer shape of the portion 12 of the core 11 and which merges into the end area 19, closed on account of the balloon design and remote from the end area 17.

The dimensions of core 11 and balloon 16 are such that, as can be seen from FIG. 2, the balloon fitted onto the core 11 bears completely on the core 11, such that the end area 17 of the balloon contacts the end area 10 of the core, and the portion 18 of the balloon 16 contacts the portion 12 of the core 11, and, finally, the end area 19 of the balloon 16 bears on the free end of the core 11. The dimensions of the balloon 16 in relation to the core 11 are chosen here such that the balloon 16 bears on the core 11 with relatively little pretensioning, in other words in a relatively unstressed state.

In order to fasten the end area 17 of the balloon 16 on the core 11, at the end area 10 of the latter, a clamping ring 20 is provided, which is fitted externally onto the balloon 16 at the end area 17 thereof. The structure thus formed is inserted with the clamping ring 20 into the recess 8 of the middle part 3, and the upper part 4 is then connected to the middle part 3, as a result of which the clamping ring 20 and therefore the core 11 and balloon 16 are held secure in the recesses 8 and 9 of middle part 3 and upper part 4. For the clamping ring 20, the recesses 8 and 9 have a seat that widens conically in the direction away from the respective free edge of the middle part 3 and upper part 4, in order to ensure a secure hold of the clamping ring 20.

The middle part 3, the upper part 4 and the lower part 5 serve to receive further operating elements of the pump 1:

A Luer check valve or lock valve 21 connected to the upper part 4 passes through an opening 22 in the upper part 4, and, as is explained in the following description of FIG. 2, has a Luer lock valve housing 23 and a Luer lock valve core 24. By way of a channel 25, the Luer lock valve 21 is in communication with a channel 26, which is formed between the upper part 4 and the middle part 3 and which communicates with the channel 13 extending through the core 11.

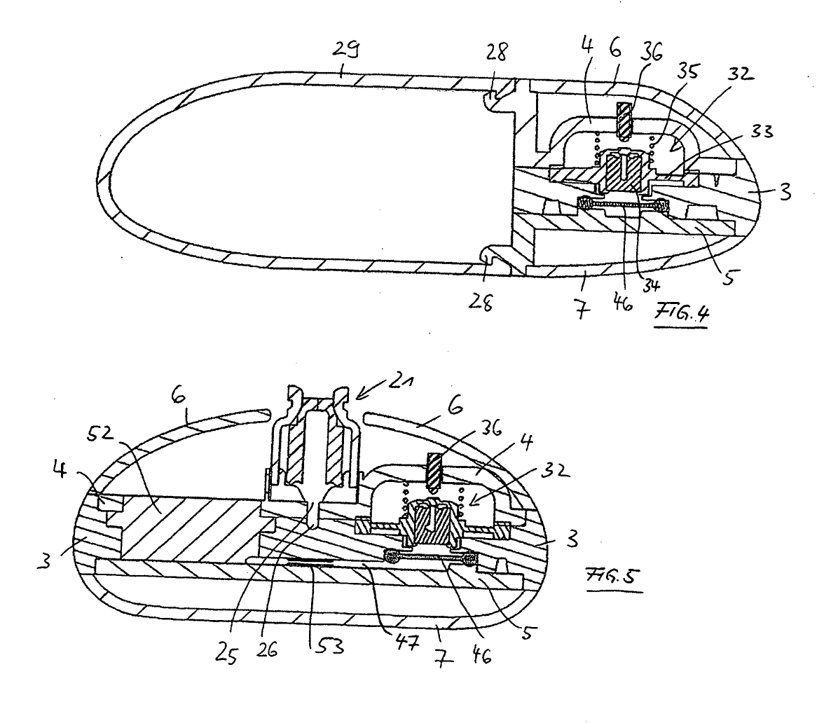

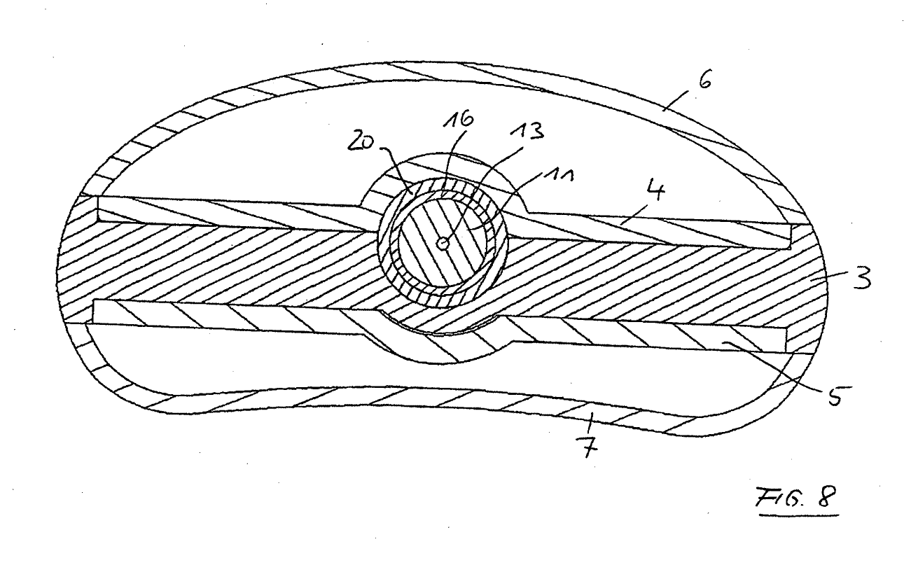

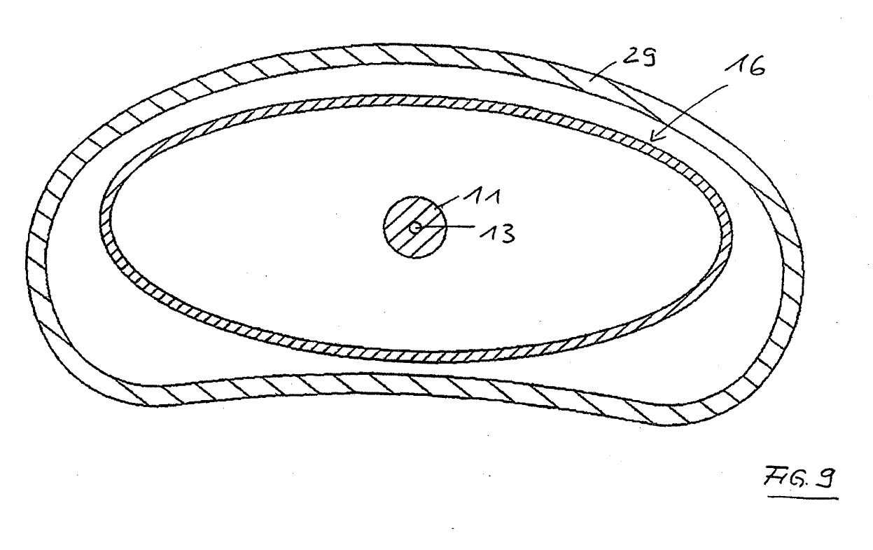

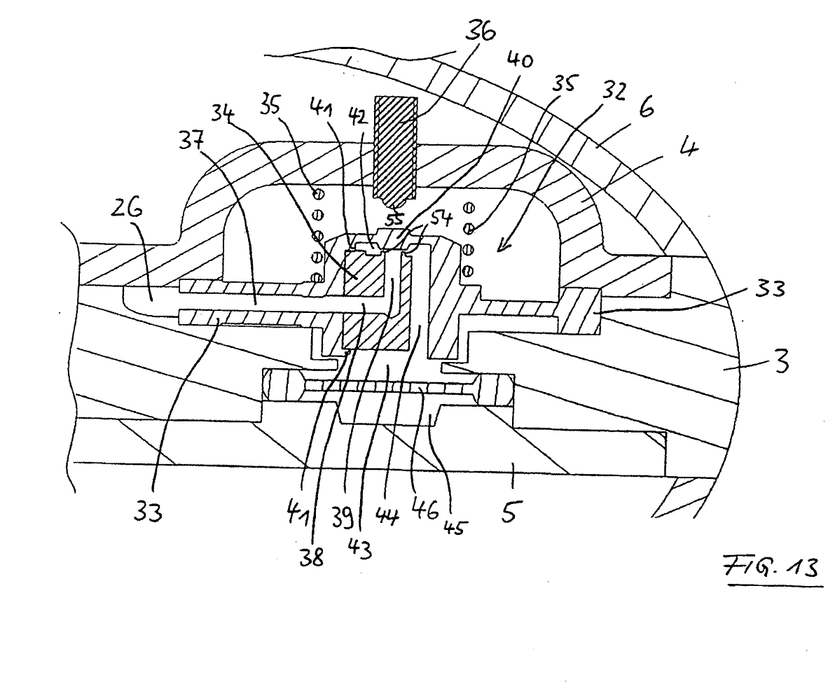

The pump is filled with liquid by way of the Luer lock valve 31 and the channels 25, 26 and 13. Starting from the unfilled state shown in FIG. 2, and with increasing delivery of liquid, the balloon 16 expands in that area not clamped by the clamping ring 20, and, when completely filled, adopts the final shape illustrated in FIG. 3. The space occupied by the liquid is designated there by reference number 27. It will be seen from FIGS. 2, 3 and 6 to 12 that, as it fills with liquid, starting from its initial state bearing on the core 11, the balloon 16 changes shape both in the longitudinal direction of the core and also in transverse directions thereof, i.e. in a first transverse direction and in a second transverse direction perpendicular thereto.

The upper part 4 and the lower part 5 are provided with locking projections 28, which serve to receive a cap 29 that is approximately kidney-shaped in cross section.

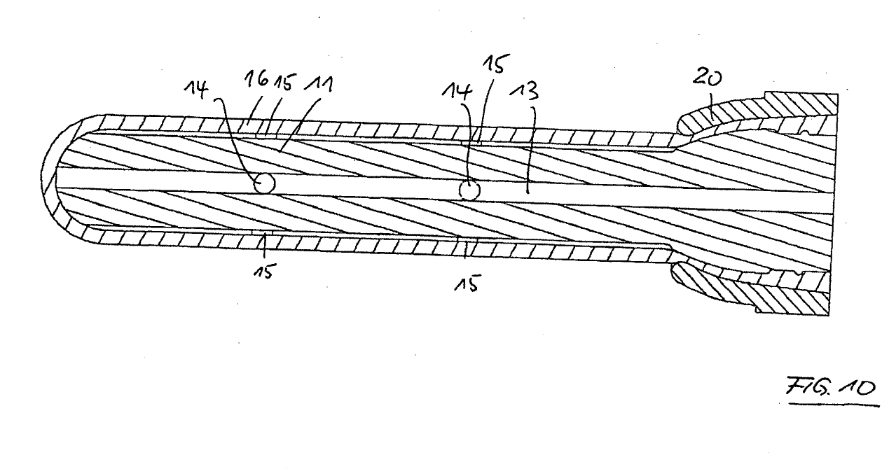

As can be seen from FIG. 9, this cap 29 has an extension in its direction of width that is substantially greater than that in the direction of its height. The width to height ratio is 2:1, for example. As can be seen from FIG. 2 for example, the length to height ratio of the cap 29 is approximately 2.5:1. The cap 29 is preferably clipped non-releasably onto the housing 2. When the balloon 16 is filled completely with liquid, it takes up as much as possible of the internal space in the cap 29.

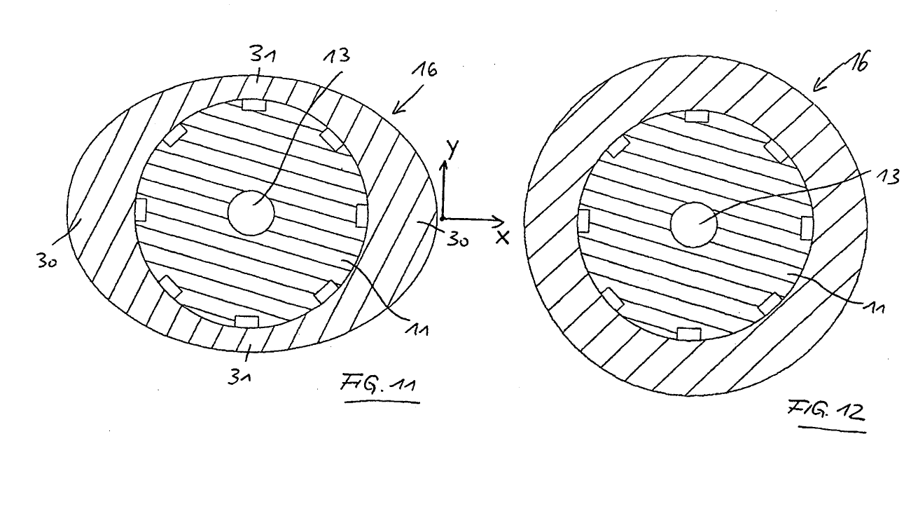

This is achieved by the fact that, as can be seen from the view in FIG. 11 showing the balloon 16 bearing on the core 11, the balloon 16 has relatively thick wall portions 30 in a first direction of extent X perpendicular to the longitudinal axis of the core 11, and it has relatively thin wall portions 31 in a second direction of extent Y perpendicular to the longitudinal axis of the core 11 and perpendicular to the first direction of extent X. Thus, when liquid is introduced into its space 27, the balloon 16 seeks to expand preferably in the direction of extent X, thereby resulting in the expanded oval cross-sectional shape illustrated in the view in FIG. 9. Overall, the pump 1 is presented as a flat functional component that can be easily worn on the body, and the balloon 16, in the state when filled with liquid, likewise adopts a flat shape adapted to the outer contour of the pump 1.

The channels 26 and 13 serve not only to deliver the liquid from the Luer lock valve 21 into the balloon 16, but also to dispense the liquid from the interior of the balloon 16 to the patient. Thus, the channel 26 is continued past the inlet point of the channel 25 to a valve 32 that is mounted in the middle part 3 and upper part 4 and that restricts the volumetric flow of liquid discharged from the balloon 16. This valve 32 is formed by an elastic valve membrane 33 held at the edge between middle part 3 and upper part 4, by a valve core 34 that interacts with the valve membrane 33, by a compression spring 35 supported on the valve membrane 33 and the upper part 4, and by an adjusting screw 36, which is mounted in a thread of the upper part 4 and can be brought into operative connection with the valve membrane 33.

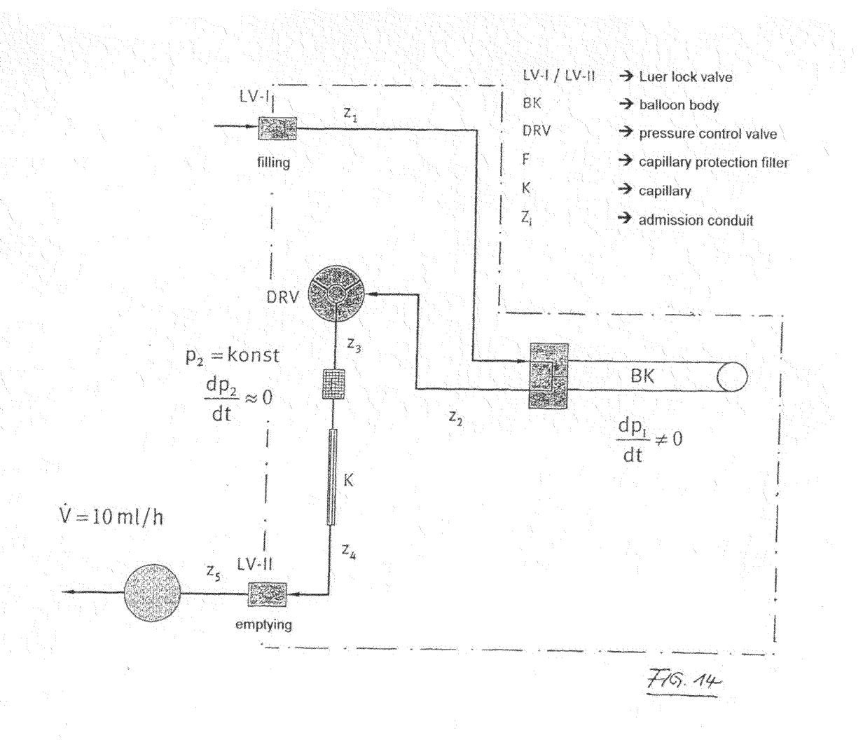

As can be seen from the detailed view in FIG. 13, the channel 26 opens into a radially extending channel 37 of the valve membrane 33 and from there into a radial channel 38 of the valve body 34, which opens into an axial channel 39 of the valve core 34. This channel 39 is open in the area of its end directed toward a reinforced portion 40 of the valve membrane 33. An abutment designed as an adjusting screw 36 is arranged on that side of the portion 40 directed away from the channel 39, which portion 40 has the function of a closure element. In principle, this abutment could also be stationary. Between the projections 41 of the valve membrane 33, the valve core 34 is held so as to be axially immovable relative to the valve membrane 33 and also non-rotatable relative to the latter.

The valve 32 is used to stop the volumetric flow in the event of too high a pressure. Two separate chambers 42 and 43 are formed in the valve and are connected to each other via a channel 44, which extends through the valve core 34 and is arranged parallel to the channel 40. The chamber 42, which lies in the direction of flow to the inlet, and therefore to the channel 26, serves as a blocking chamber. The chamber 43 lies in the direction of flow to the outlet 45. To filter the liquid dispensed through the valve 32, a filter 46 is provided which is clamped at the edge between the middle part 3 and the lower part 5. Starting from the chamber 43 and the outlet 45, the liquid passes to a channel 47 (FIG. 5) in flow communication with the outlet 45, and from there to a Luer lock attachment 48 held between the middle part 3 and the lower part 5. A Luer lock connector 49, provided with a hose 50 leading to the patient, can be connected to the Luer lock attachment 48.

As can be seen from the view in FIG. 5, a glass capillary 53 is fitted into the channel 47. This glass capillary constitutes a flow restrictor, which is able to restrict the volumetric flow passing through the channel 47 out of the pump, since the flow restrictor has a smaller cross section than the channel 37 lying in the inlet. By selecting various flow restrictors, it is possible to set various constant flow rates, as long as the pressure at the inlet does not drop below a defined value. In principle, the cross-sectional area of flow of the inlet is greater than the cross-sectional area of flow of the outlet. Of course, the flow restrictor can be designed other than in the form of a glass capillary. For example, it is entirely conceivable to provide downstream of the valve, in the outlet of the pump, a meander chip that restricts the through-flow.

Because of the stated diameters of the channels that connect the space 27 of the balloon to the valve 32, and the diameter of the channels arranged behind the valve 32 with the flow restrictor 53, the resistance that the channel 47 with flow restrictor 53 sets against the outflow of liquid from the housing 2 is greater than the resistance made to the liquid flowing into the valve 32.

In an initial state, the valve membrane 33 is located in the position shown in FIG. 13, in which the valve membrane 33 bears largely on the middle part 3, without requiring any action of the compression spring 35. Because of the positioning of the valve core 34 relative to the portion 40 of the valve membrane 33, a small gap is provided between the portion 40 and an encircling and therefore annular projection 54 of the valve core 34. This projection 54 encloses the channel 43. Accordingly, liquid flows through the channel 13 of the core 11 and through the adjoining housing channel 26 into the channel 37 of the valve membrane 33 and from there into the channels 38 and 39 of the valve core 34. From the channel 39 of the valve core 34, the liquid flows through the gap formed between the projection 54 and the portion 40 of the valve membrane 33, and into the chamber 42 located there, and from the chamber 42 through the channel 44 between valve membrane 33 and valve core 34 to the chamber 43, passes the filter 46 and travels through the outlet 45 to the channel 47 with the flow restrictor 53. If a higher liquid pressure is established in the inlet, thus also in the channel 39, without a greater volumetric flow being able to issue from the pump as a result of the flow restrictor 43, this has the result that the valve membrane 33, which is clamped in the edge area between the middle part 3 and the upper part 4, deforms in the central area in the direction of the adjusting screw 36 with the abutment function, specifically counter to the force of the compression spring 35. When the valve membrane 34 with its portion 40 comes up against the projection 55 of the adjusting screw 36 directed toward the portion 40, the portion 40 makes contact there with the adjusting screw 36, such that, since the valve membrane 33 cannot move any farther up in the direction of the upper shell, the portion 40 is pressed against the projection 54 of the valve core 34 and thus closes the flow through the channel 39. As the liquid flows out through the flow restrictor 53, the pressure in the chamber 43 decreases, with the result that the membrane, by virtue of its own elasticity, moves back again in the direction of its initial state according to FIG. 13, such that the portion 40 disengages from its contact with the adjusting screw 36, and the flow gap between the projection 54 and the portion 40 is again freed. Depending on the pressure prevailing in the balloon 16, this state can be obtained only when the initial position of the valve membrane 33 is reached, as shown in FIG. 13, or even earlier, in other words with the valve membrane 33 still deflected. The adjusting screw 36 serves to modify the opening and closing behavior of the valve 32. The further the screw frees the adjustment path of the valve membrane, the greater is the secondary pressure in the valve. In principle, it is not necessary to provide the compression spring 35. It is of advantage when greater pressures are intended to be dealt with by the pump 1 and, accordingly, the elastic restoring behavior of the valve membrane 33 is not sufficient to move it into the initial position according to FIG. 13.

With the valve 32, the volumetric flow of liquid is therefore restricted as a function of the pressure prevailing in the balloon 16, and the volumetric flow of liquid is maintained substantially constant via the flow restrictor 53. In principle, the liquid pump could be modified by providing only a device for maintaining substantially constant the volumetric flow of liquid dispensed from the elastic element, or only a device for restricting the volumetric flow of liquid dispensed from the elastic element.

Before using the mechanically operated liquid pump, liquid is delivered through the Luer lock valve 21, as a result of which the liquid passes into the balloon 16, and the filling level of the balloon can be read off through the transparent cap 29 on the basis of the markings 51 which are arranged in the transverse direction of the cap and which are a reference for the transverse expansion of the balloon as a function of its state of filling. After the pump 1 has been filled and the pump has been attached to the patient via the hose 50, liquid is dispensed out of the pump through the valve 32, with elastic pretensioning of the expanded balloon 16, and this is done until the balloon has been completely emptied and bears on the core 11.

The particularly simple design of the described liquid pump allows it to be used in a variety of different ways. The user is able to operate the pump anywhere, and immediately, without long start-up times. It can be used carried around by the user, or used in one place, specifically in all normal life situations in or outside the field of medicine. The pump can be used in a sterile state and requires minimal operating/handling effort. Because of the simple construction of the small number of component parts, the pump is inexpensive to produce. This is a condition for its being able to be used particularly in outpatient care, and in financially weak markets. The low weight of the pump permits its use in accident and emergency situations, in field hospitals and in disaster areas. Some or all of the functional elements of the pump are exchangeable. The pump is suitable for short or long dispensing times, for example, in the case of a balloon with a capacity of 25 ml, for a flow rate of 2.5 ml per hour, that is to say a running time of 10 hours. It is of course possible to use other balloons with other volumes, for example 10 ml, 50 ml, 100 ml or 150 ml. The running time can be much longer, for example up to 24 hours. Although flow rates of >1000 ml per hour are entirely possible, a flow rate of 0.5 to 10 ml per hour is considered the preferred option.

According to the illustrative embodiment, a balloon is described which is produced by injection molding and serves as a container for the medicament solution and as a pressure reservoir. The balloon has a defined contour in cross section and in expansion, for filling flat housing spaces and for avoiding pressure peaks. It is radially and/or axially pretensioned on a one-part or multi-part core, in order to increase the restoring forces. One end of the balloon is sealed off in an airtight manner over the core and fixed in position by a clamping ring with a form fit. The balloon is freely movable in the axial and radial directions during filling and emptying, being elastically deformable and able to move in a manner free from friction inside the cap.

The pump 1 can additionally be provided with a bolus reservoir. In FIG. 1, the provision of such a bolus is indicated by reference number 52. The pump can be converted to this extent, as and when required.

FIG. 14 is a diagram illustrating how the above-described mechanically operated pump works and showing the physical parameters. The contour of the pump is indicated by the dot-and-dash line.

In the foregoing description, it will be readily appreciated by those skilled in the art that modifications may be made to the invention without departing from the concepts disclosed herein. Such modifications are to be considered as included in the following claims, unless these claims by their language expressly state otherwise.

The invention claimed is as follows:

1-14. (canceled)

15. A mechanically operated liquid pump for medical and nutrient liquids and the like, comprising:
a housing member;
an expandable elastic member shaped to store and dispense a liquid therefrom, and being disposed generally within said housing member;
a closeable inlet member regulating the flow of the liquid traveling into said elastic member;
an outlet member routing the liquid traveling out of said elastic member;
a flow regulator member communicating with said outlet member, and maintaining a substantially constant volumetric flow of the liquid dispensed from said elastic member; wherein
said elastic member comprises a balloon having an open end portion thereof and an opposite distal portion thereof, and including
a core member mounted in said housing member and being inserted through said open end portion and into said balloon; and
a fastening member sealingly mounting said open end portion of said balloon on one of said core member and said housing member, whereby in a relatively unstressed state, said distal portion of said balloon bears on said core member.

16. A pump as set forth in claim 15, wherein:
said balloon is constructed from silicone.

17. A pump as set forth in claim 16, wherein:
said balloon has relatively thick wall portions along a first axis disposed generally perpendicular to the longitudinal axis of said core, and relative thin wall portions along a second axis disposed generally perpendicular to the longitudinal axis of said core and generally perpendicular to said first axis.

18. A pump as set forth in claim 17, wherein:
said balloon, when filled with the liquid, has a generally flat shape.

19. A pump as set forth in claim 18, including:
a cap connected with said housing, and having a kidney shape in transverse cross section to the longitudinal axis of said core.

20. A pump as set forth in claim 19, wherein:
said cap has a width/height ratio in the range of 1.5:1 to 3:1.

21. A pump as set forth in claim 20, wherein:
said core has a generally cylindrical shape, and includes a channel extending through said core and communicating with said inlet and outlet members.

22. A pump as set forth in claim 21, wherein:
said channel extends along the longitudinal center axis of said core.

23. A pump as set forth in claim 22, wherein:
said balloon, in said unstressed shape, has a generally cylindrical shape with dimensions corresponding substantially to the dimensions of said core, and disposed in substantial rotational symmetry therewith.

24. A pump as set forth in claim 23, wherein:
said core has a length/diameter ratio in the range of 3:1 to 8:1.

25. A pump as set forth in claim 24, wherein:
said balloon has an internal volume in the range of 10 to 150 milliliters.

26. A pump as set forth in claim 25, wherein:
said balloon includes an injection molded construction.

27. A pump as set forth in claim 26, wherein:
said balloon is configured to bear completely on said core in said unstressed state.

28. A pump as set forth in claim 27, wherein:
said core includes a sleeve over which said open end portion of said balloon is positioned; and
said fastening member comprises a clamp which engages the open end of said balloon and attaches the same to said sleeve.

29. A pump as set forth in claim 15, wherein:
said balloon has relatively thick wall portions along a first axis disposed generally perpendicular to the longitudinal axis of said core, and relative thin wall portions along a second axis disposed generally perpendicular to the longitudinal axis of said core and generally perpendicular to said first axis.

30. A pump as set forth in claim 15, wherein:
said balloon, when filled with the liquid, has a generally flat shape.

31. A pump as set forth in claim 15, including:
a cap connected with said housing, and having a kidney shape in transverse cross section to the longitudinal axis of said core.

32. A pump as set forth in claim 31, wherein:
said cap has a width/height ratio in the range of 1.5:1 to 3:1.

33. A pump as set forth in claim 15, wherein:
said core has a generally cylindrical shape, and includes a channel extending through said core and communicating with said inlet and outlet members.

34. A pump as set forth in claim 33, wherein:
said channel extends along the longitudinal center axis of said core.

35. A pump as set forth in claim 15, wherein:
said balloon, in said unstressed shape, has a generally cylindrical shape with dimensions corresponding substantially to the dimensions of said core, and disposed in substantial rotational symmetry therewith.

36. A pump as set forth in claim 15, wherein:
said core has a length/diameter ratio in the range of 3:1 to 8:1.

37. A pump as set forth in claim 15, wherein:
said balloon has an internal volume in the range of 10 to 150 milliliters.

38. A pump as set forth in claim 25, wherein:
said balloon includes an injection molded construction.

39. A pump as set forth in claim 15, wherein:
said balloon is configured to bear completely on said core in said unstressed state.

40. A pump as set forth in claim 15, wherein:
said core includes a sleeve over which said open end portion of said balloon is positioned; and
said fastening member comprises a clamp which engages the open end of said balloon and attaches the same to said sleeve.
